(19) United States
(12) Patent Application Publication
(10) Pub. No.: US 2021/0393546 A1
(43) Pub. Date: Dec. 23, 2021

(54) COMPOSITIONS FOR PREVENTING AND TREATING VIRAL INFECTIONS

(71) Applicants: Maria CRISLER, Rock Springs, WY (US); Emma DiPONIO, Dorado, PR (US); J. Phillip KENNEDY, Grand Rapids, MI (US)

(72) Inventors: Maria CRISLER, Rock Springs, WY (US); Emma DiPONIO, Dorado, PR (US); J. Phillip KENNEDY, Grand Rapids, MI (US)

(73) Assignee: SHAMAN NATURALS, LLC, Rock Springs, WY (US)

(21) Appl. No.: 16/906,350

(22) Filed: Jun. 19, 2020

(51) Int. Cl. A61K 31/05; A61K 9/00; A61K 45/06; A61K 47/26; A61K 47/36; A61K 47/02; A61K 47/12; A61K 47/24; A61K 47/18; A61K 36/185; A61P 31/22

(52) U.S. Cl. CPC A61K 31/05; A61K 9/0014; A61K 9/0053; A61K 45/06; A61K 47/26; A61P 31/22; A61K 47/02; A61K 47/12; A61K 47/24; A61K 47/183; A61K 36/185; A61K 47/36

(57) ABSTRACT

Embodiments of the invention are directed to compositions containing cannabinoid, cannabidiol, cannabidiol isomer, or cannabidiol analog and combinations thereof for treating viral infections, and methods for treating viral infections by topically or orally administering compositions containing cannabinoid, cannabidiol, or cannabidiol analog to the patient in need of treatment.

### CROSS-REFERENCE TO RELATED APPLICATIONS

This application claims priority from U.S. Provisional No. 62/680,037 entitled “Compositions for Preventing and Treating Viral Infections” filed Jun. 20, 2019, and U.S. Provisional No. 63/010,290 entitled “Compositions for Preventing and Treating Viral Infections” filed Apr. 15, 2020, the contents of each of which are hereby incorporated by reference in its entirety.

### GOVERNMENT INTERESTS

Not applicable

### PARTIES TO A JOINT RESEARCH AGREEMENT

Not applicable

### INCORPORATION OF MATERIAL ON COMPACT DISC

Not applicable

### BACKGROUND

Not applicable

### SUMMARY OF THE INVENTION

Various embodiments include a composition containing a cannabinoid and a brassinosteroid, or combinations thereof, and in some embodiments, a pharmaceutical excipient, diluent, reagent, and the like and combinations thereof. In some embodiments, the cannabinoid may have a concentration of about 0.5 wt. % to about 50 wt. %, relative to the total amount of the compositions. In some embodiments, the brassinosteroid may have a concentration of about 0.01 wt. % to about 5 wt. %, relative to the total amount of the composition. In some embodiments, the composition may contain an amino acid, peptide, protein, or combination thereof, and in some embodiments, the amino acid, peptide, or protein may have a concentration of about 0.01 wt. % to about 5 wt. %, relative to the total amount of the composition. In some embodiments, the composition may further contain an antioxidant, and in some embodiments, the antioxidant may have a concentration of about 0.01 wt. % to about 5 wt. %, relative to the total amount of the topical composition. In some embodiments, the composition may include an anti-inflammatory agent, and in some embodiments, the anti-inflammatory agent may have a concentration of about 0.01 wt. % to about 5 wt. %, relative to the total amount of the topical composition. In some embodiments, the composition may include a mineral or mineral salt, and in some embodiments, the mineral or mineral salt may have a concentration of about 0.01 wt. % to about 5 wt. %, relative to the total amount of the topical composition. In some embodiments, the compositions may include cyanobacteria or green algae, and in some embodiments, the cyanobacteria or green algae may have a concentration of about 0.01 wt. % to about 5 wt. %, relative to the total amount of the topical composition. In various embodiments, the composition may be formulated as a cream, lotion, salve, liniment, ointment, gel, paste, tonic, tincture, unguent, soap, shampoo, topical, oral, pills, tablet, capsule, lip balm, or combinations thereof.

Further embodiments are directed to methods for treating viral infections by administering any of the compositions described above to a subject in need of treatment. In some embodiments, administering can be carried out by oral administering, topical administering, or combinations thereof. In some embodiments, the viral infection can be caused by viruses include, for example, herpes simplex virus 1 (HSV-1), herpes simplex virus 2 (HSV-2), varicella zoster virus (VSV/HHV-3), Epstein-Barr virus (EBV/HHV-4), cytomegalovirus (CMV/HHV-5), human herpesvirus type 6 (HBLV/HHV-6), human herpesvirus type 7 (HHV-7), human herpesvirus type 8 (KSHV/HHV-8), human papillomavirus, infectious mononucleosis, shingles, chickenpox, poxviruses, molluscum contagiosum, lymphoma, rhinoviruses, and enteroviruses, and the subjects may exhibits symptoms such as, but not limited to, herpes blisters, blisters, sores, fever blisters, herpes related lesions, fever, warts, common warts, palmoplantar warts, flat warts, recurrent warts, recalcitrant warts, treatment naïve warts, epidermodysplasia verruciformis related warts, anogenital warts. viral shedding, and viral replication. Various embodiments are directed to topical and oral formulations containing cannabinoids, cannabidiols, cannabidiol isomers, cannabidiol analogs, or combinations thereof a carrier, excipient, diluent, reagent, or combinations thereof, and an additive or combination of additives and methods for preventing and treating viral infections by topically or orally administering a composition containing cannabinoids, cannabidiols, cannabidiol isomers, cannabidiol analogs, or combinations thereof. In certain embodiments, the symptoms prevented and treated may be caused by herpes simplex virus type 1 (HSV-1) or type 2 (HSV-2).

Some embodiments are directed to topical formulations. The formulations may include cannabidiols, cannabidiol isomers, cannabidiol analogs, or combinations thereof and a carrier, excipient, diluent, reagent, or combinations thereof. And in some embodiments, such formulations may further include one or more brassinosteroid agents, antibiotic agents, antiviral agents, antioxidants, peptides, amino acid co-factors, vitamins, essential amino acids, non-essential amino acids, trace minerals, barrier agent, drying agent, hydrating agent, and combinations thereof. Such formulations may interrupt or prevent herpes virus replication and may provide a reduction in herpetic symptoms such as, but not limited to herpes blisters, sores, fever blisters, and viral shedding.

Some embodiments are directed to oral formulations. The formulations may include cannabidiols, cannabidiol isomers, cannabidiol analogs, or combinations thereof and a carrier, excipient, diluent, reagent, or combinations thereof. And in some embodiments, such formulations may further include one or more brassinosteroid agents, antibiotic agents, antiviral agents, antioxidants, peptides, amino acid co-factors, vitamins, essential amino acids, non-essential amino acids, trace minerals, barrier agent, drying agent, hydrating agent, and combinations thereof. Such formulations may interrupt or prevent herpes virus replication and may provide a reduction in herpetic symptoms such as, but not limited to herpes blisters, sores, fever blisters, and viral shedding.

### DESCRIPTION OF THE DRAWINGS

Examples of the specific embodiments are illustrated in the accompanying drawings. While the invention will be described in conjunction with these specific embodiments, it will be understood that it is not intended to limit the invention to such specific embodiments. On the contrary, it is intended to cover alternatives, modifications, and equivalents as may be included within the spirit and scope of the invention. In the following description, numerous specific details are set forth in order to provide a thorough understanding of the present invention. The present invention may be practiced without some or all of these specific details. In other instances, well known process operations have not been described in details so as to not unnecessarily obscure the present invention

The patent or application file contains at least one drawing executed in color. Copies of this patent or patent application publication with color drawing(s) will be provided by the office upon request and payment of the necessary fee.

FIG. 1 are images showing patient progression during treatment using the compositions of the invention. FIG. 1A shows an untreated HSV-1 breakout. FIG. 1B shows the patient 2 hrs after initial administration. FIG. 1C shows the patient 14 hrs after beginning treatment, and FIG. 1D shows the patient 37 hours after beginning treatment.

FIG. 2 are images showing patient progression during treatment using the compositions of the invention. FIG. 2A shows an untreated HSV-1 breakout. FIG. 2B shows the patient 4 hrs after initial administration. FIG. 2C shows the patient 24 hrs after beginning treatment.

FIG. 3 are images showing patient progression during treatment using the compositions of the invention. FIG. 3A shows an untreated Zoster shingles breakout. FIG. 3B shows the patient 55 hrs after administration. FIG. 3C shows the patient 72 hrs after administration, and FIG. 3D shows the patient 7 days hours after beginning treatment.

### DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 ml to 8 ml is stated, 2 ml, 3 ml, 4 ml, 5 ml, 6 ml, and 7 ml are also intended to be explicitly disclosed, as well as the range of values greater than or equal to 1 ml and the range of values less than or equal to 8 ml.

All percentages, parts and ratios are based upon the total weight of the topical compositions and all measurements made are at about 25° C., unless otherwise specified.

The singular forms “a,” “an,” and “the” include plural referents unless the context clearly dictates otherwise. Thus, for example, reference to a “polymer” includes a single polymer as well as two or more of the same or different polymers; reference to an “excipient” includes a single excipient as well as two or more of the same or different excipients, and the like.

The word “about” when immediately preceding a numerical value means a range of plus or minus 10% of that value, e.g, “about 50” means 45 to 55, “about 25,000” means 22,500 to 27,500, etc, unless the context of the disclosure indicates otherwise, or is inconsistent with such an interpretation. For example, in a list of numerical values such as “about 49, about 50, about 55, “about 50” means a range extending to less than half the interval(s) between the preceding and subsequent values, e.g, more than 49.5 to less than 52.5. Furthermore, the phrases “less than about” a value or “greater than about” a value should be understood in view of the definition of the term “about” provided herein.

The terms “administer,” “administering” or “administration” as used herein refer to either directly administering a compound (also referred to as an agent of interest) or pharmaceutically acceptable salt of the compound (agent of interest) or a composition to a subject.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical, cosmetic or other agent across a tissue layer such as the stratum corneum or stratum spinosum.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. In embodiments or claims where the term comprising is used as the transition phrase, such embodiments can also be envisioned with replacement of the term “comprising” with the terms “consisting of” or “consisting essentially of.”

The term “disorder” is used in this disclosure to mean, and is used interchangeably with the terms disease, condition, symptom, or illness, unless otherwise indicated.

The terms “effective amount” and “therapeutically effective amount” are used interchangeably in this disclosure and refer to an amount of a compound that, when administered to a subject, is capable of reducing a symptom of a disorder in a subject or enhance the texture, appearance, color, sensation, or hydration of the intended tissue treatment area. The actual amount which comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The phrase “pharmaceutically acceptable” or “cosmetically acceptable” is employed herein to refer to those agents of interest/compounds, salts, compositions, dosage forms, etc, which are—within the scope of sound medical judgment—suitable for use in contact with the tissues of human beings and/or other mammals without excessive toxicity, irritation, allergic response, or other problem or complication, commensurate with a reasonable benefit/risk ratio. In some aspects, pharmaceutically acceptable means approved by a regulatory agency of the federal or a state government, or listed in the U.S. Pharmacopeia or other generally recognized pharmacopeia for use in mammals (e.g, animals), and more particularly, in humans.

The term “salts” as used herein embraces pharmaceutically acceptable salts commonly used to form alkali metal salts of free acids and to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid. Non-limiting examples of such inorganic acids are hydrochloric, hydrobromic, hydroiodic, nitric, carbonic, sulfuric, and phosphoric acid. Appropriate organic acids can be selected from aliphatic, cycloaliphatic, aromatic, aryl aliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, 3-hydroxybutyric, galactaric and galacturonic acid.

The term “patient” and “subject” are interchangeable and may be taken to mean any living organism which may be treated with compounds of the present invention. As such, the terms “patient” and “subject” may include, but is not limited to, any non-human mammal, primate or human. In some embodiments, the “patient” or “subject” is a mammal, such as mice, rats, other rodents, rabbits, dogs, cats, swine, cattle, sheep, horses, primates, or humans. In some embodiments, the patient or subject is an adult, child or infant. In some embodiments, the patient or subject is a human.

The term “treating” is used herein, for instance, in reference to methods of treating a skin disorder or a systemic condition, and generally includes the administration of a compound or composition which reduces the frequency of, or delays the onset of, symptoms of a medical condition or enhance the texture, appearance, color, sensation, or hydration of the intended tissue treatment area of the tissue surface in a subject relative to a subject not receiving the compound or composition. This can include reversing, reducing, or arresting the symptoms, clinical signs, and underlying pathology of a condition in a manner to improve or stabilize a subject's condition.

The term “herb” is used herein, for instance, in reference to plants that in certain embodiments and delivered by appropriate methods have a therapeutic or medicinal purpose, such as, but not limited to river mint, eucalyptus, wattle, cocoa, plants of the family cannabaceae, plants containing cannabinoids, and plants containing cannabinoid precursors and analogs.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, analogs, compounds, ligands, structures, or groups thereof, or any members of a claimed group, less than the full measure of this disclosure can be claimed for any reason. Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Various embodiments are directed to compositions for treating viral infections containing cannabinoids and methods for using such compositions to treat, prevent, and ameliorate viral infections. In certain embodiments, the composition may include brassinosteroids. Particular embodiments are directed to methods for treating viral infections by administering the compositions described above. Such compositions may be administered orally or topically, or in combination thereof, in therapeutically effective doses. The compositions and methods of the invention may reduce outbreaks, symptoms, viral shedding, and proliferation of viral infections by reducing viral loads and targeting sites of viral replication.

The compositions of the invention have a general antiviral effect. Therefore, viruses treated using the compositions and methods of embodiments are not limited. For example, the viral infections may be caused by herpes simplex virus 1 (HSV-1), herpes simplex virus 2 (HSV-2), varicella zoster virus (VSV/HHV-3), Epstein-Barr virus (EBV/HHV-4), cytomegalovirus (CMV/HHV-5), human herpesvirus type 6 (HBLV/HHV-6), human herpesvirus type 7 (HHV-7), human herpesvirus type 8 (KSHV/HHV-8), human papillomavirus, infectious mononucleosis, shingles, chickenpox, poxviruses, molluscum contagiosum, lymphoma, rhinoviruses, and enteroviruses, among others. In some embodiments, the viral infections may be caused by, for example, lassa virus, lymphocytic choriomeningitis virus (LCMV), junin virus, machupo virus, guanarito virus, sabia virus, severe acute respiratory syndrome (SARS) virus, murine hepatitis virus (MHV), human coronavirus, COVID-19, bovine coronavirus, canine coronavirus, feline infectious peritonitis virus, ebola virus, marburg virus, influenza A virus, influenza B virus, influenza C virus, measles virus, mumps virus, canine distemper virus, newcastle disease virus, human immunodeficiency virus 1 (HIV-1), human immunodeficiency virus 2 (HIV-2), human T-cell lymphotrophic virus 1 (HTLV-1), human T-cell lymphotrophic virus 2 (HTLV-2), human intracisternal A-type particle 1 (HIAP-1), human intracisternal A-type particle 2 (HIAP-2), and the like.

Symptoms associated with such viruses vary and may include, but are not limited to, herpes blisters, sores, fever blisters, viral shedding, warts, common warts, palmoplantar warts, flat warts, recurrent warts, recalcitrant warts, treatment naïve warts, epidermodysplasia verruciformis related warts, anogenital warts, condyloma accuminatum, cervical dysplasias or neoplasias, e.g., cervical intraepithelial neoplasia (CIN); Herpesvirus related lesions including those induced by, for example, HHV-1 (HSV-1), HHV-2 (HSV-2), HHV-3 (varicella-zoster virus), Poxvirus induced lesions caused by, for example, chicken pox, Herpes zoster, shingles, molluscum contagiosum, orf, callus, cutaneous horns, corns, acrochordons, fibroepithelial polyps, prurigo nodularis, actinic keratoses, squamous cell carcinoma, keratoacanthoma, basal cell carcinoma, cutaneous lymphomas and benign lymphocytic infiltrates & hyperplasias of the skin, clear cell acanthoma, large cell acanthoma, epidermolytic acanthoma, porokeratosis, hyperkeratosis, keratosis pilaris, lichenoid keratosis, acanthosis, acanthosis nigricans, confluent and reticulated papillomatosis, nevi, including e.g., dermal nevi, epidermal nevi, compound nevi, ILVEN (inflammatory linear verrucous epidermal nevi), nevus sebaceous, nevus comedonicus, and the like; acne, e.g., comedonal acne, inflammatory acne, papular acne, pustular acne, cystic acne; cysts, e.g., epidermoid cysts, milia, trichilemmal cysts, follicular cysts, proliferating cysts, dermoid cysts, pilonidal cysts, apocrine cysts, eccrine cysts, sebaceous cysts, mucous cysts, myxoid cysts, ganglion cysts, synovial cysts, vellus hair cysts, steatocystoma, hidrocystoma; adnexal neoplasms e.g., trichofolliculoma, fibrofolliculoma, perifollicular fibroma, trichodiscoma, nevus sebaceous, chondroid syringoma, trichoepithelioma, trichoblastoma, desmoplastic trichoepithelioma, pilomatricoma, pilomatrical carcinoma, tricholemmoma, trichelemmal carcinoma, tumor of the follicular infundibulum, tricoadenoma, proliferating pilar tumor, sebaceous hyperplasia, sebaceous adenoma, sebaceous epithelioma, sebaceous carcinoma, syringoma, poroma, hidradenoma, apocrine hidradenoma, spiradenoma, cylindroma, eccrine nevus (eccrine hamartoma), papillary adenoma, papillary adenocarcinoma; benign melanocytic proliferations or neoplasms e.g., ephilides, café-au-lait macules, Becker's melanosis, lentigines, solar lentigines, lentigo simplex, mucosal melanocytic lesions, Mongolian spots, Nevus of Ota, blue nevus, common acquired melanocytic nevi (nevocellular nevus, “moles”), congenital nevi, nevus spilus, recurrent nevi; vascular and perivascular neoplasms and reactive hyperplasias e.g., hemangiomas, cherry angiomas, hobnail hemangiomas (targeted hemosiderotic hemangiomas), tufted angiomas, hemangioendotheliomas, angiolymphoid hyperplasia with eosinophilia (ALHE), Glomus tumors (glomangiomas), hemangiopericytomas; cutaneous neural and neuroendocrine neoplasms e.g., neuromas, Schwannomas, neurofibromas, nerve sheath tumor, nerve sheath myxoma, neurothekeoma, granular cell tumor; fibrotic and fibrohistiocytic proliferations e.g., acrochordons, fibroepithelial polyps, fibromas, fibrous papules, angiofibromas, pearly penile papules, periungual fibromas, dermatofibromas, fibrokeratomas, sclerotic or pleomorphic fibromas, connective tissue nevi; cutaneous scars, hyperplasias, keloids, rosacea, cutaneous fungal, dermatophyte & mold infections, onychomycosis, hyperpigmentation, rhytides, psoriasis, malignant melanoma, seborrheic keratosis, seborrheic keratosis variants including e.g., dermatosis papulosis nigra, inverted follicular keratosis/keratoma warty dyskeratosis/warty dyskeratoma, acrokeratosis verruciformis, stucco keratosis; or a combination thereof. Compositions of embodiments may produce a reduction in viral symptoms, while improving the condition of the affected skin zones following treatment.

The cannabinoids of such embodiments include any of a broad class of compounds that are known to interact with cannabinoid receptors, and encompass endocannabinoids (produced naturally in the body by animals), the phytocannabinoids (found in cannabis and some other plants), and synthetic cannabinoids (manufactured artificially). Example cannabinoids include, but are not limited to, tetrahydropyran analogs, such as, Δ⁹-tetrahydrocannabinol, Δ⁸-tetrahydrocannabinol, 6,6,9-trimythel-3-pentyl-6H-dibenzo[b,d]pyran-1-ol, 3-(1,1-dimethylheptyl)-6,6a7,8,10,10a-hexahydro-1-1 hydroxy-6,6-dimethyl-9H-dibezo[b,d]pyran-9-ol, (−)-(3 S,4 S)-7-hydroxy-delta-6-tetrahydrocannabinol-1,1-dimethylheptyl, (+)-(3 S,4S)-7-hydroxy-Δ-6-tetrahydrocannabinol, and Δ⁸-tetrahydrocannabinol-11-oic acid, piperidine analogs, such as, (−)-(6S,6aR,9R,10aR)-5,6,6a,7,8,9,10,10a-octahydro-6-methyl-1-3-[(R)-1-methyl-4-phenylbutoxy]-1,9 phenanthridinediol 1-acetate), aminoalkylindole analogs, such as, (R)-(+)-[2,3-dihydro-5-methyl-3-(4-morpholinylm-ethyl)-pyrrolo[1,2,3,-de]-1,4-benzoxazin-6-yl-1-naphthelenyl-methanone, open pyran-ring analogs, such as, 243-methyl-6-(1-methylethenyl-2-cyclohexen-1-yl]-5-pentyl-1,3-benzendi-ol, and 4-(1,1-dimethylheptyl)-2,3′-dihydroxy-6′-α-(3-hydroxypropyl)-1′,-2′,3 ‘,4’,5′,6′-hexahydrobiphenyl, lipophilic alkylamides, such as, dodeca-2E,4E,8Z,10E/Z-tetraenoic-acid-isobutylamide, cannabinoid mimetics, salts, solvates, metabolites, and metabolic precursors of these compounds and combinations thereof. In some embodiments, the cannabinoids may be derived plants including hemp, Echinacea purpurea, Echinacea angustifolia, Acmella oleracea, Helichrysum umbraculigerum, Radula marginata, and combinations thereof and oils made from these plants, and in other embodiments, the cannabinoids may be manufactured or chemically synthesized.

The compositions of various embodiments can include any number of cannabinoids in various concentrations; however, in certain embodiments, the cannabinoid may be cannabidiol (2-(6-isopropenyl-3-methyl-5-cyclohexen-1-yl)-5-pentyl-1,3-benzenediol). Cannabidiol has 7 double bonds and 30 stereoisomers. Embodiments include compositions containing each stereoisomer individually and compositions containing a combination of these stereoisomers. In particular embodiments, the compositions used in the methods of embodiments and the compositions of embodiments may include high concentrations of cannabidiol. For example, in some embodiments, cannabidiol may be about 30 w/v % to about 100 w/v % of the cannabinoids in the composition, and in other embodiments cannabidiol may be about 50 w/v % to about 100 w/v %, about 75 w/v % to about 100 w/v %, about 80 w/v % to about 100 w/v %, about 90 w/v % to about 100 w/v % of the cannabinoids in the composition.

Cannabidiol can be obtained by cold-pressing industrial hemp with trace amounts of THC. Cannabidiol in this present invention is provided as a natural constituent of hemp oil.

In some embodiments, the cannabinoids in the composition may be cannabidiol analogs. The term “cannabidiol analogs” refers to synthetically produced compounds that are structurally similar, but not structurally identical, to cannabidiol. Various cannabidiol analogs are known in the art and embodiments encompass such cannabidiol analogs. For example, PCT Publication WO2017/132526 and U.S. Pat. No. 6,630,507, which are each hereby incorporated by reference in their entireties, describes various analogs of cannabidiol. In some embodiments, the analogs of cannabidiol may be of general Formula I:

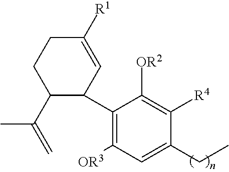

where R¹ is hydrogen, methyl, linear or branched C₂-C₁₀ alkyl, linear or branched C₂-C₁₀ alkenyl, linear or branched C₂-C₁₀ substituted alkyl, linear or branched C₂-C₁₀ substituted alkenyl, R² and R³ are each, individually, hydrogen, methyl, linear or branched C₂-C₁₀ alkyl, linear or branched C₂-C₁₀ substituted alkyl, linear or branched C₂-C₁₀ alkenyl, linear or branched C₂-C₁₀ substituted alkenyl, linear or branched C₂-C₁₀ acyl, linear or branched C₂-C₁₀ substituted acyl, an amine or amino acid, amino acid ester, R⁴ is hydrogen, substituted or unsubstituted alkyl, carboxyl, alkoxy, aryl, aryloxy, arylalkyl, halo or amino, and n may an integer of 2 to 10 and the like and salts and solvates thereof. In some embodiments, R² and R³ may, independently, be a linear or branched, substituted or unsubstituted C₂-C₁₀ acyl having a carboxylic acid terminus thereby producing a dicarboxylic acid, and salts thereof. Like cannabidiol, cannabidiol analogs can have various isomers. Embodiments include all isomers of the such cannabidiol analogs.

In some embodiments, cannabidiol analogs, such as those described above may be combined with cannabidiol, to produce a mixture of cannabidiol and cannabidiol analogs. Thus, as used herein the term “cannabidiol” encompasses cannabidiol, cannabidiol analogs, and the various isomers of cannabidiol and cannabidiol analogs.

The compositions of various embodiments can include up to about 50% (w/w) cannabidiol, cannabidiol analogs, isomers of cannabidiol, cannabidiol analogs, and combinations thereof (collectively, “cannabidiol”), and in some embodiments, the compositions may include from about 50% (w/w) to about 0.5% (w/w), about 30% (w/w) to about 1% (w/w), about 20% (w/w) to about 1% (w/w), about 20% (w/w) to about 5% (w/w) cannabidiol, or any range of or individual concentration encompassed by these example ranges. In particular embodiments, the composition may include about 15% (w/w) to about 10% (w/w) cannabidiol.

In certain embodiments, the cannabidiol of embodiments described above may be cannabidolic acid (“CBDA”). Without wishing to be bound by theory, CBDA may exhibit improved hydrophilicity over other isomers of cannabidiol, which may allow for improved solubility and delivery of CBDA to the skin. The CBDA may be modified, partially digested, or otherwise acted upon by enzymes in the skin to produce for example cannabidiol (CBD), which may be the active form cannabidiol in the composition. Thus, CBDA may act as a prodrug in some embodiments of the invention. Other cannabidiol analogs or isomers may produce a similar effect and are encompassed by prodrug embodiments of the invention.

The cannabidiol in the compositions of embodiments of the invention may be 100% cannabidiol, or oils, solvents, and emulsions containing cannabidiol. For example, in some embodiments, the compositions of the invention may include cannabidiol derived from hempseed oil. Hempseed oil is generally manufactured from varieties of Cannabis sativa that do not contain significant amounts of tetrahydrocannabinol (THC), the psychoactive element present in the cannabis plant. This manufacturing process typically includes cleaning the seed to 99.99% before pressing the oil. Hempseed oil generally also contains omega-6 and omega-3 fatty acids. For example, about 30-35% of the weight of hempseed oil are essential fatty acids (EFAs), i.e., linoleic acid, omega-6 (LA, 55%), a-linolenic acid, omega-3 (ALA, 22%), γ-linolenic acid, omega-6 (GLA, 1-4%), and stearidonic acid, omega-3 (SDA, 0-2%). Thus, the compositions of some embodiments may contain fatty acids such as omega-6 and omega-3 fatty acids.

Oils include cannabidiol oil and various plant derived oils containing cannabidiol, such as, hempseed oil, Echinacea purpurea, Echinacea angustifolia, Acmella oleracea, Helichrysum umbraculigerum, Radula marginata, and the like. In some embodiments, cannabidiol isolated from such plants or made synthetically may be formulated with an oil such as, for example, olive oil, grapeseed oil, tea tree oil, almond oil, avocado oil, sesame oil, evening primrose oil, sunflower oil, kukui nut oil, jojoba oil, walnut oil, peanut oil, pecan oil, macadamia nut oil, coconut oil, and the like and combinations thereof.

In some embodiments, the topical compositions may further include a brassinosteroid or combinations of brassinosteroids. Brassinosteroids are a group of compounds related to brassinolide, a C28 steroid with a lactone B-ring structure. Brassinosteroids include, but are not limited to, 24(S) ethylbrassinone analogs, (22R,23R,24S)-2alpha, 3 alpha, 5 alpha,22,23-pentahydroxy-stigmastan-6-one, (22R,23R,24 S)-3beta-bromo-5alpha,22,23-trihydroxystigmastan-6-one, (22 S,23 S,24 S)-2alpha,3 alpha,22,23-tetrahydroxy-5 alpha, stigmastan-6-one, (22R,23R,24 S)-3beta-acetoxy-22,23-dihydroxy-5alpha-cholestan-6-one, (22S,23 S,24S)-3beta-bromo-22,23-dihydroxy-5alpha-cholestan-6-one, (22S,23 S,24S)-3beta-bromo-5alpha,22,23-trihydroxy-stigmastan-6-one, and (22S,23 S)-3-bromo-5α,22,23-trihydroxystigmastan-6-one.

The amount of brassinosteroid in the topical formulation is not limited, so long as it is a therapeutically effective amount. In some embodiments, the brassinosteroid may have a concentration of about 0.01 wt. % to about 5 wt. %, relative to the total amount of the composition, about 0.1 wt. % to about 1 wt %, relative to the total amount of the composition, or any range or individual value encompassed by these example ranges.

Without wishing to be bound by theory, the combination of cannabinoid and brassinosteroid may provide enhanced antiviral activity compared to the antiviral effect of these compounds individually, to the extent either cannabinoids or brassinosteroids exhibit antiviral activity. Thus, the compositions of the invention are capable of reducing viral load and improving symptoms related to the viral infection more quickly than either component alone.

In some embodiments, the compositions may further include an anti-inflammatory compound such as hyaluronic acid, curcumin, glutathione, methotrexate, tofacitinib, 6-mercaptopurine, azathioprine sulphasalazine, mesalazine, olsalazine chloroquinine/hydroxychloroquinine, penicillamine, aurothiomalate (intramuscular and oral), azathioprine, cochicine, corticosteroids (oral, inhaled, and local injection), a beta-2 adrenoreceptor agonist (salbutamol, terbutaline, salmeteral), a xanthine (theophylline, aminophylline), cromoglycate, nedocromil, ketotifen, ipratropium and oxitropium, cyclosporin, FK506, rapamycin, mycophenolate mofetil, leflunomide, an NSAID (e.g. ibuprofen), a corticosteroid (e. g. prednisolone), a phosphodiesterase inhibitor, an adensosine agonist, an antithrombotic agent, a complement inhibitor, an adrenergic agent, an agent that interferes with signalling by proinflammatory cytokines such as TNF or IL-1 (e.g., a NIK, IKK, p38 or MAP kinase inhibitor), an IL-1 converting enzyme inhibitor, a T-cell signalling inhibitor (e.g. a kinase inhibitor), a metalloproteinase inhibitor, sulfasalazine, a 6-mercaptopurine, an angiotensin converting enzyme inhibitor, a soluble cytokine receptor (e.g. soluble p55 or p75 TNF receptors and the derivatives p75TNFRigG (etanercept) and p55TNFRigG (Lenercept), siL-1RI, siL-1RII, siL-6R), an antiinflammatory cytokine (e.g. IL-4, IL-10, IL-11, IL-13 and TGF), celecoxib, folic acid, hydroxychloroquine sulfate, rofecoxib, etanercept, infliximab, adalimumab, certolizumab, tocilizumab, abatacept, naproxen, valdecoxib, sulfasalazine, methylprednisolone, meloxicam, methylprednisolone acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone HCl, hydrocodone bitartrate/apap, diclofenac sodium/misoprostol, fentanyl, anakinra, tramadol HCl, salsalate, sulindac, cyanocobalamin/fa/pyridoxine, acetaminophen, alendronate sodium, prednisolone, cortisone, betamethasone, morphine sulfate, lidocaine hydrochloride, indomethacin, glucosamine sulf/chondroitin, amitriptyline HCl, sulfadiazine, oxycodone HCV acetaminophen, olopatadine HCl misoprostol, naproxen sodium, omeprazole, cyclophosphamide, rituximab, IL-1 TRAP, MRA, CTLA4-IG, IL-18 BP, anti-IL-12, Anti-IL1S, BIRB-796, SCIO-469, VX-702, AMG-548, VX-740, Roflumilast, IC-485, CDC-801, S1PI agonists (such as FTY720), a PKC family inhibitor (e.g. Ruboxistaurin or AEB-071) or Mesopram, budenoside; epidermal growth factor; a corticosteroid; cyclosporin, sulfasalazine; an aminosalicylate; 6-mercaptopurine; azathioprine; metronidazole; a lipoxygenase inhibitor; mesalamine; olsalazine; balsalazide; an antioxidant; a thromboxane inhibitor; an IL-1 receptor antagonist; an anti-IL-1 monoclonal antibody; an anti-IL-6 monoclonal antibody; a growth factor; an elastase inhibitor; a pyridinyl-imidazole compound; an antibody to or antagonist of other human cytokines or growth factors (e.g. TNF, LT, IL-1, IL-2, IL-6, IL-7, IL-8, IL-12, IL-15, IL-16, IL-23, EMAP-II, GM-CSF, FGF, and PDGF); a cell surface molecule (e.g. CD2, CD3, CD4, CD8, CD25, CD28, CD30, CD40, CD45, CD69, or CD90 or their ligands); methotrexate; cyclosporine; FK506; rapamycin; mycophenolate mofetil; leflunomide; an NSAID (e.g. ibuprofen); a corticosteroid (e.g. prednisolone); a phosphodiesterase inhibitor; an adenosine agonist; an antithrombotic agent; a complement inhibitor; an adrenergic agent; an agent that interferes with signalling by proinflammatory cytokines such as TNF 5 or IL-1 (e.g. a NIK, IKK, or MAP kinase inhibitor); an IL-1 converting enzyme inhibitor; a TNF converting enzyme inhibitor; a T-cell signalling inhibitor such as kinase inhibitors; a metalloproteinase inhibitor; sulfasalazine; azathioprine; a 6-mercaptopurine; an angiotensin converting enzyme inhibitor; a soluble cytokine receptor (e.g. soluble p55 or p75 TNF receptors, siL-1RI, siL-1RII, siL-6R), an antiinflammatory cytokine (e.g. IL-4, IL-10, IL-11, IL-13 or TGF), therapeutic agents that target an intrinsic checkpoint blockade, such as, for example, the gene encoding Cytokine-inducible SH₂-containing protein (CISH), antibody BGB-A317, Nivolumab, or Pembrolizumab, atezolizumab, avelumab, durvalumab, ipilimumab, and the like and combinations thereof.

The amount of anti-inflammatory agent is not limited and includes any therapeutically effective amount. For example, in some embodiments, the amount of anti-inflammatory agent may be about 0.01 wt. % to about 5 wt %, relative to the total amount of the composition, about 0.1 wt. % to about 1 wt %, relative to the total amount of the formulation, or any range or individual concentration encompassed by these example ranges.

In some embodiments, the compositions may further include an antibiotic. The type of antibiotic is not limited, and can be, for example, subtilosin, ampicillin, bacampicillin, carbenicillin indanyl, mezlocillin, piperacillin, ticarcillin, amoxicillin-clavulanic acid, ampicillin-sulbactam, benzylpenicillin, cloxacillin, dicloxacillin, methicillin, oxacillin, penicillin G, penicillin V, piperacillin tazobactam, ticarcillin clavulanic acid, nafcillin, procaine penicillin, cefadroxil, cefazolin, cephalexin, cephalothin, cephapirin, cephradine, cefaclor, cefamandol, cefonicid, cefotetan, cefoxitin, cefprozil, ceftmetazole, cefuroxime, loracarbef cefdinir, ceftibuten, cefoperazone, cefixime, cefotaxime, cefpodoxime proxetil, ceftazidime, ceftizoxime, ceftriaxone, cefepime, azithromycin, clarithromycin, clindamycin, dirithromycin, erythromycin, lincomycin, troleandomycin, cinoxacin, ciprofloxacin, enoxacin, gatifloxacin, grepafloxacin, levofloxacin, lomefloxacin, moxifloxacin, nalidixic acid, norfloxacin, ofloxacin, sparfloxacin, trovafloxacin, oxolinic acid, gemifloxacin, perfloxacin, imipenem-cilastatin, meropenem, and aztreonam.

The amount of the antibiotic in the compositions is not limited, and includes any therapeutically effective amount. For example, the antibiotic may have a concentration of about 0.01 wt. % to about 5 wt %, relative to the total amount of the composition, about 0.1 wt. % to about 1 wt %, relative to the total amount of the composition, or any range or individual concentration encompassed by these example ranges.

In some embodiments, the composition may further include a plant extract such as, but not limited to, phytochemicals. Phytochemicals can include chemical compounds that naturally occur in plants such as flavonoids or bioflavonoids. Bioflavonoids can include flavonoids, isoflavanoids, neoflavanoids, and anthoxanthins flavones (e.g., luteolin, apigenin, and tangeritin), flavonols (e.g., quercetin, kaempferol, myricetin, fisetin, galangin, isorhamnetin, pachypodol, rhamnazin, pyranoflavonols, and furanoflavonols), flavones (e.g., hesperetin, naringenin, eriodictyol, and homoeriodictyol), flavanonol (e.g., taxifolin and dihydrokamferol), flavans (e.g., flavan-3-ols, anthocyanidins, and isoflavinoids). In other embodiments, upregulating compounds comprise extracts derived from edible plants. For example, the plant extracts may include glucoraphnin or sulforaphanederived derived from broccoli, catechin, epicatachin, and proanthocyanidins from grapes, grape seed extract, milk thistle, and blueberries, and other related compounds. In certain embodiments, the plant extract may include alpha lipoic acid, resveratrol, curcumin, EGCG, Olivol®, rutin, quercetin, hesperetin, and the like and combinations thereof.

In some embodiments, the compositions may further include a secondary antiviral agent. The antiviral compound is not limited and includes, for example, subtilosin, adamantane agents, chemokine receptor agonists, integrase strand transfer inhibitors, neuraminidase inhibitors, NNRTI agents, NSSA inhibitors, ribavirin, valacyclovir, acyclovir, famciclovir, ribavirin, valganciclovir, ribavirin, ganciclovir, cidofovir, fomivirsen, sofosbuvir, enfuvirtide, foscarnet, letermovir, ibalizumab, and baloxavir marboxil. Such antiviral agents may be included in the compositions of embodiments in the form of nanoparticles, nanoclusters, or nanostrands, or nanofibers.

The amount of the antiviral agent in the compositions is not limited, and includes any therapeutically effective amount. For example, the antiviral agent may have a concentration of about 0.01 wt. % to about 5 wt %, relative to the total amount of the composition, about 0.1 wt. % to about 1 wt %, relative to the total amount of the composition, or any range or individual concentration encompassed by these example ranges.

In some embodiments of the present invention, compositions may further contain a mineral, mineral salt, or combinations thereof. Such minerals are not limited, and can include selenium, sulfur, zinc, iron, chlorine, cobalt, copper, manganese, molybdenum, and iodine.

The amount of the mineral or mineral salts in the topical formulation is not limited, and includes any therapeutically effective amount. For example, the mineral or mineral salt may have a concentration of about 0.01 wt. % to about 5 wt %, relative to the total amount of the composition, about 0.1 wt. % to about 1 wt %, relative to the total amount of the composition, or any range or individual concentration encompassed by these example ranges.

In some embodiments of the present invention, the compositions may further inlcude a vitamin or a combination of vitamins. Vitamins are organic molecules that are essential nutrients that organisms need to sustain proper biological function and metabolism. The vitamins encompassed by the invention are not limited, and can be, for example, vitamin A, vitamin B₁, vitamin B₂, vitamin B₃, vitamin B₄, vitamin B₅, vitamin B₆, vitamin B₇, vitamin B₈, vitamin B₉, vitamin B₁₀, vitamin B₁₁, vitamin B₁₂, vitamin C, vitamin D, vitamin E, and vitamin K.

The amount of the vitamin in the topical formulation is not limited, and can be any therapeutically effective amount. For example, the vitamin may have a concentration of about 0.01 wt. % to about 5 wt %, relative to the total amount of the composition, about 0.1 wt. % to about 1 wt %, relative to the total amount of the composition, or any range or individual concentration encompassed by these example ranges.

In some embodiments, the compositions may further contain amino acids, peptides, or combinations thereof. Amino acids are organic compounds that combine through peptide bond formation to form peptides and proteins. Amino acids can chemically combine through peptide bond formation to form dipeptides, tripeptides, tetrapeptides, oligopeptides, polypeptides, peptides, and proteins. Amino acids are the building blocks for living organisms. The human body uses amino acids to break down food, grow, repair body tissue, and perform other necessary biological processes. The amino acid is not limited, and can be at least one member selected from the group consisting of L-arginine, D-arginine, L-histidine, D-histidine, L-lysine, D-lysine, L-aspartic acid, D-aspartic acid, L-glutamic acid, D-glutamic acid, D-serine, L-serine, D-threonine, L-threonine, D-asparagine, L-asparagine, L-glutamine, D-glutamine, L-cystine, D-cysteine, L-selenocysteine, D-selenocysteine, L-glycine, D-glycine, L-proline, D-proline, L-alanine, D-alanine, L-valine, D-valine, L-isoleucine, D-isoleucine, L-leucine, D-leucine, L-methionine, D-methionine, L-phenylalanine, D-phenylalanine, L-tyrosine, D-tyrosine, L-tryptophan, D-tryptophan.

The amount of the amino acids, peptides, or combinations thereof in the composition is not limited, and includes any therapeutically effective amount. For example, the amino acid, peptides, or combinations thereof may have a concentration of about 0.01 wt. % to about 5 wt %, relative to the total amount of the composition, about 0.1 wt. % to about 1 wt %, relative to the total amount of the composition, or any range or individual concentration encompassed by these example ranges.

In some embodiments, the compositions may further include cyanobacteria, green algae, or combinations thereof, such as, aphanizomenon flos-aquae (E3Live™), Arthrospira platensis, Synechocystis, Spirulina, photoautotrophic cyanobacteria, and combinations thereof. Cyanobacteria are a phylum of bacteria that include photosynthetic prokaryotes able to produce oxygen. Cyanobacteria may possess the ability to produce substances that serve as anti-inflammatory agents and combat infection in humans. Some cyanobacteria have been shown to trigger substantial movement of natural killer cells (NKCs), which are cells that provide rapid response to virus-infected cells.

The amount of cyanobacteria, green algae, or combinations thereof in the compositions is not limited, and includes any therapeutically effective amount. For example, cyanobacteria, green algae, or combinations thereof in the compositions may have a concentration of about 0.01 wt. % to about 5 wt %, relative to the total amount of the composition, about 0.1 wt. % to about 1 wt %, relative to the total amount of the composition, or any range or individual concentration encompassed by these example ranges.

Creams refer to semi-solid emulsions of oil and water in approximately equal proportions. They are divided into two types: oil-in-water (O/W) creams, composed of small droplets of oil dispersed in a continuous phase; and water-in-oil (W/O) creams, composed of small droplets of water dispersed in a continuous oily phase. Creams can provide a barrier to protect the skin. This may be a physical barrier or a chemical barrier as with UV-absorbing compounds. To aid in the retention of moisture (especially water-in-oil creams), creams are usually used for a variety of purposes including cleansing, emollient effects, and as a vehicle for drug substances such as local anesthetics, anti-inflammatories (NSAIDs or corticosteroids), hormones, antibiotics, antifungals or counter-irritants.

Liniments or balms are topical formulations that are of a similar viscosity to lotions and less viscous than an ointment or cream. Liniments are generally applied with friction by rubbing the liniment into the skin. Liniments typically are formulated from alcohol, acetone, or similar quickly evaporating solvents and may contain counterirritant aromatic chemical compounds such as methyl salicylate, benzoin resin, or capsaicin.

Ointments are compositions in which oil and water are provided in a ratio of from 7:1 to 2:1, from 5:1 to 3:1, or 4:1. Ointments are generally formulated using oils, waxes, water, alcohols, petroleum products, water, and other agents to prepare formulations with various viscosities and solvent properties. Commonly used formulations include oleaginous base (White Ointment), absorption base, W/O emulsion base (Cold Cream type base), O/W emulsion base (Hydrophilic Ointment), water soluble base, in addition to others. These preparations are used to dissolve or suspend substances or products with medicinal or cosmetic value.

Lotions are low- to medium-viscosity topical preparation. Most lotions are oil-in-water emulsions containing an emulsifier such as cetyl alcohol to prevent separation of these two phases. Lotions can include fragrances, glycerol, petroleum jelly, dyes, preservatives, proteins and stabilizing agents.

In some embodiments, the formulations can be in the form of a soap, which are formulations that comprise a salt of a fatty acid. Soaps are mainly used as surfactants for washing, bathing, and cleaning, but they are also used in textile spinning and are important components of lubricants. Soaps for cleansing are usually obtained by treating vegetable or animal oils and fats with a strongly alkaline solution. Fats and oils are composed of triglycerides; three molecules of fatty acids are attached to a single molecule of glycerol. The alkaline solution, which is often called lye (although the term “lye soap” refers almost exclusively to soaps made with sodium hydroxide), is believed to promote a chemical reaction known as saponification. In saponification, the fats are first hydrolyzed into free fatty acids, which then combine with the alkali to form crude soap. Glycerol (glycerine) is usually liberated and is either left in or washed out and recovered as a useful byproduct, depending on the process employed.

In some embodiments, the composition can be in the form of a shampoo, which is a hair care product used for the removal of oils, dirt, skin particles, dandruff, environmental pollutants and other contaminant particles that gradually build up in hair. A goal may be to remove the unwanted build-up without stripping out so much sebum as to make hair unmanageable.

In some embodiments, the composition can be in the form of a tincture. Tinctures are herbal extracts that provide a method for oral administration of an herbal component or components to a subject in need of treatment. Tinctures are prepared by mixing an herb or herbs or components and combinations thereof with a suitable solvent wherein a component or components of an herb or herbs or combinations thereof are extracted into a solvent in which the component or components of the herb are reasonably soluble. Suitable tincture solvents in the present invention include pharmacologically acceptable solvents such as organic solvents, water based solvents, alcohols, and other orally administrable solvents such as, but not limited to, water, purified water, preserved water, vegetable glycerin, propylene carbonate, 3-methoxy-3-methyl-1-butanol (MMB), polyethylene glycol, glycerol, rice bran oil, and combinations thereof.

In some embodiments, the composition can be in the form of a tonic. Tonics are extracts that provide a method for oral administration of an herbal component or components to a subject in need of treatment. Tonics are prepared by mixing an herb or herbs or components and combinations thereof with a suitable solvent wherein a component or components of an herb or herbs or combinations thereof are extracted into a solvent by aid of heating, often heat necessary such that the solvent reaches its boiling temperature, in which the component or components of the herb are reasonably soluble. Suitable tonic solvents in the present invention include pharmacologically acceptable solvents such as organic solvents, water based solvents, alcohols, and other orally administrable solvents such as, but not limited to, water, purified water, preserved water, vegetable glycerin, propylene carbonate, 3-methoxy-3-methyl-1-butanol (MMB), polyethylene glycol, glycerol, rice bran oil, and combinations thereof.

In some embodiments, the composition can be in the form of a tablet. Tablets are pharmaceutical oral dosage forms of a medicament or medicaments that are formed by molding or compression. Such embodiments may include a medicament or medicaments and may further include suitable excipients such as, but not limited to, diluents, binders, granulating agents, gildants, lubricants, disintegrants, sweeteners, and pigments. Tablets in the present invention may also be coated with a pigment to increase the visual appearance of the tablet, to increase the identifiability of the tablet, to increase the ease with which the tablet is orally administered, to make the tablet more easily swallowed, to control the release of the medicament or medicaments, or to make the tablet more resistant to environmental degradation factors, or a combination or combinations thereof.

In some embodiments, the composition can be in the form of a capsule. Capsules generally fall within the class of either hard-shelled capsules or soft-shelled capsules, but need not be restricted to either class. Hard shelled capsules generally, but need not necessarily, contain dry, powdered, or granular components while soft-shelled capsules primarily, but need not necessarily, contain oils or medicaments or combinations thereof.

Tables 1-3 below provide specific examples of formulations encompassed by the invention. The compositions of Table 1 include Retinol and/or hyaluronic acid, which are optional ingredients, but that may be beneficial in certain applications.

TABLE 1

| CBD or analog or combinations thereof | N/A | |
|---|---|---|
| Brassinosteroid or analog or combinations thereof | 30-100 | μM |
| Subtilosin | 40-100 | mg/mL |
| Retinol | 0.5% | (w/w) |
| Hyaluronic acid | 0.5-1.5% | (w/w) |
| Glutathione | 800-1500 | mg |
| Curcumin | N/A | |
| Vitamin C | 350-1000 | mg |
| L-lysine | 1000-3000 | mg |
| Selenium | 55-125 | mg |
| Sulfur | 1000-1500 | mg |
| Zinc | 10-15 | mg |

The compositions of Table 2 do not include Retinol or hyaluronic acid, but are effective for treating viral infections.

TABLE 2

| CBD or analog or combinations thereof | N/A | |
|---|---|---|
| Brassinosteroid or analog or combinations thereof | 30-100 | μM |
| Subtilosin | 40-100 | mg/mL |
| E3Live | N/A | |
| Glutathione | 800-1500 | mg |
| Curcumin | N/A | |
| Vitamin C | 350-1000 | mg |
| L-lysine | 1000-3000 | mg |
| Selenium | 55-125 | mg |
| Sulfur | 1000-1500 | mg |
| Zinc | 10-15 | mg |

The compositions of Table 3 Quercetin, but do not include Retinol or hyaluronic acid. Such compositions are effective at treating viral infections and are beneficial in certain applications.

TABLE 3

| CBD or analog or combinations thereof | N/A | |
|---|---|---|
| Brassinosteroid or analog or combinations thereof | 30-100 | μM |
| Quercetin | 1.5-150 | mg/mL |
| E3Live | N/A | |
| Glutathione | 800-1500 | mg |
| Curcumin | N/A | |
| Vitamin C | 350-1000 | mg |
| L-lysine | 1000-3000 | mg |
| Selenium | 55-125 | mg |
| Sulfur | 1000-1500 | mg |
| Zinc | 10-15 | mg |

Various embodiments are directed methods for preventing, inhibiting proliferation of, or treating viral infections by administering any of the compositions described above including cannabinoids, and in some embodiments, brassinosteroids to the subject in need of treatment. Administering can be carried out topically or orally, and in some embodiments, a course of treatment may include both topical and oral administration either concurrently or sequentially. For example, in some embodiments, a topical composition may be administered between breakout of blisters or sores caused by HSV-1 or HSV-2 to reduce the likelihood of a breakout. In the event of a breakout, topical administration may be continued and concurrent oral administration may be carried out, topical administration may be replaced by oral administration, or the dosage of the topical composition administered may be increased.

The viruses treated and symptoms associated with these viral infections include any of those described above. In particular embodiments, the viral infection may be caused by HSV-1 or HSV-2. The dermatological symptoms may include, but are not limited to herpes blisters, sores, fever blisters, and viral shedding, and combinations thereof. Antiviral formulations may produce a reduction in dermatological viral symptoms, while improving the condition of the affected skin zones following treatment. The compositions of various embodiments can be used to aid healing of viral induced tissue wounds, and in some embodiments, the dermatological disease may be associated with wounds or chronic wounds.

Another embodiment of the present invention is a method of making the topical formulation in the form of a cream, which comprises (i) dispersing lake/powder into mineral oil or silicone oil to obtain an oil phase; (ii) dispersing an emulsifier, a thickener; and a stabilizer into water in a separate vessel to obtain an aqueous phase; (iii) blending the oil phase and the aqueous phase to form an emulsion; and (iv) dispersing an active ingredient such as a Cannabis derived botanical drug product into at least one of the oil phase, the aqueous phase, and the emulsion. In some embodiments, the method further comprises heating during at least one of (i) dispersing lake/powder into mineral oil or silicone oil to obtain an oil phase and (ii) dispersing an emulsifier, a thickener; and a stabilizer into water in a separate vessel to obtain an aqueous phase. Temperatures of this heating are not particularly limited, so long as the oil phase and the aqueous phase result from the dispersing.

Another embodiment of the present invention is a method of making the topical formulation in the form of a lotion, which comprises mixing an oil phase comprising hemp oil with an emulsifier and with an aqueous phase to form a mixture and heating said mixture at a temperature of from 45 and 85° C. to form an aqueous emulsion. Emulsifiers include, but are not limited to, cetyl alcohol, stearic acid, and a mixture thereof. The water phase comprises a stabilizing agent such as VEEGUM® or CARBOPOL®.

Another embodiment of the present invention is a method of making the topical formulation in the form of a shampoo, which comprises combining a surfactant, most often sodium lauryl sulfate and/or sodium laureth sulfate with a co-surfactant, most often cocamidopropyl betaine, in an aqueous phase and mixing the aqueous phase to form a thick, viscous liquid. Preferred methods further comprise adding other ingredients, such as salt (sodium chloride), a preservative, and fragrance, to the aqueous phase.

Another embodiment of the present invention is a method of treating manifestations of dermatological conditions caused by a viral infection, which comprises applying a therapeutically effective amount of the topical formulation, according to the present invention, to skin affected with a dermatological condition. Non-limiting examples of targeted dermatological conditions include herpes blisters, sores, fever blisters, and viral shedding.

Another embodiment of the present invention is a method for preventing manifestations of dermatological conditions caused by a viral infection, which comprises oral administration of a therapeutically effective amount of the oral formulation, according to the present invention, to a subject in need of treatment. Non-limiting examples of methods of oral administration are in the form of liquids, tinctures, tonics, pills, capsules, and tablets taken orally. Non-limiting examples of targeted dermatological conditions include herpes blisters, sores, fever blisters, and viral shedding.

Unless indicated otherwise, the term “therapeutically effective amount” is not particularly limited, so long as at least one of THC and CBD is present in an amount effective for treating the dermatological disease. Preferably, the therapeutically effective amount of at least one of THC and CBD is from 2 to 100 milligrams per kilogram, more preferably from 2 to 50 milligrams per kilogram, and more preferably from 2 to 25 milligrams per kilogram. The most preferred therapeutically effective amount of THC and/or CBD in the topical formulation according to the present invention is from 2 to 10 milligrams per kilogram. All rational numbers between the preceding minima and maxima are included in the ranges.

Without wishing to be bound by theory, the compositions of various embodiments may inhibit manifestation, replication, and proliferation of viruses and aid in healing viral induced injury and inflammation in subjects in need of treatment, including those with viral infection.

### EXAMPLES

Although the present invention has been described in considerable detail with reference to certain preferred embodiments thereof, other versions are possible. Therefore, the spirit and scope of the appended claims should not be limited to the description and the preferred versions contained within this specification. Various aspects of the present invention will be illustrated with reference to the following non-limiting examples.

### Example 1

### Breakout Balm

The following formulation was prepared as a balm:

TABLE 4

| Ingredient | Amount | |
|---|---|---|
| Brassinosteroids | 383 uL of 0.001 mg/mL solution | |
| Glutathione | 0.67 | g |
| Lysine | 2 | g |
| Ascorbyl Palmitate | 0.5 | g |
| Zinc Citrate | 571 uL of 0.001 mg/mL solution | |
| Cannabidiol | 0.5 | g |
| Selenomethionine | 2 | mg |
| Hyaluronic acid | 0.2 | g |
| Distilled water | 3 | mL |
| Polysorbate-20 | 3 | mL |
| Polysorbate-80 | 0.8 | mL |

The formulation above may further include 20 g of one or more of grapeseed oil, yellow beeswax, peppermint oil, organic coconut oil, sweet basil leaf oil, black pepper oil, roman chamomile flower oil, german chamomile flower oil, cinnamon leaf oil, citronella oil, eucalyptus leaf oil, helichrysum flower oil, ginger root oil, pink grapefruit peel oil, juniper berry oil, lemongrass oil, pine needle oil, ravensara oil, rosemary leaf oil, spearmint oil, wild oregano oil, organic cypress oil, fennel oil, lemon peel oil, lavender flower oil, and the like, which can be used to modify the consistency of the formulation and add flavor.

The various ingredients were weighed and combined. The zinc citrate solution was added to these dry ingredients. Distilled water, Polysorbate-20, and Polysorbate-80 were then added to this mixture. The solution was heated until liquid, approximately 80° C. The solution was added to containers at 80° C. and allowed to cool to room temperature.

When essential oil is added to the formulation, the oil is added to the ingredient mixture and stirred at approximately 80° C. until the liquid has a uniform consistency. This solution is added to containers at 80° C. and allowed to cool to room temperature.

### Tincture

The following formulation was prepared as 30 mL of an oral tincture.

TABLE 5

| Ingredient | Amount | |
|---|---|---|
| Brassinosteroids | 576 uL of 0.001 mg/mL solution | |
| Glutathione | 1 | g |
| Lysine | 1 | g |
| Ascorbyl Palmitate | 0.5 | g |
| Zinc Citrate | 861 uL of 0.001 mg/mL solution | |
| Cannabidiol | 0.5 | g |
| Selenomethionine | 3 | mg |
| BioQ | 40 | mg |
| Distilled water | 10 | mL |
| Polysorbate-20 | 3 | mL |
| Polysorbate-80 | 0.8 | mL |

The glutathione, lysine, zinc citrate, brassinosteroids, selenomethionine, and BioQ were dissolved into water. The ascorbyl palmitate and cannabidiol were dissolved into 14 mL sweet almond oil. The aqueous and organic solutions were combined to create a biphasic mixture. The polysorbate was added and the resulting mixture was stirred until a uniform opaque yellow mixture at room temperature.

### Preventive Balm

The following formulation was prepared as 30 mL of a preventive balm.

TABLE 6

| Ingredient | Amount | |
|---|---|---|
| Glutathione | 0.67 | g |
| Lysine | 0.67 | g |
| Ascorbyl Palmitate | 0.5 | g |
| Zinc Citrate | 571 uL of 0.001 mg/mL solution | |
| Cannabidiol | 0.5 | g |
| Selenomethionine | 2 | mg |
| Hyaluronic acid | 0.2 | g |
| Distilled water | 10 | mL |
| Polysorbate-20 | 3 | mL |
| Polysorbate-80 | 0.8 | mL |

The formulation above may further include 20 g of one or more of grapeseed oil, yellow beeswax, peppermint oil, organic coconut oil, sweet basil leaf oil, black pepper oil, roman chamomile flower oil, german chamomile flower oil, cinnamon leaf oil, citronella oil, eucalyptus leaf oil, helichrysum flower oil, ginger root oil, pink grapefruit peel oil, juniper berry oil, lemongrass oil, pine needle oil, ravensara oil, rosemary leaf oil, spearmint oil, wild oregano oil, organic cypress oil, fennel oil, lemon peel oil, lavender flower oil, and the like, which can be used to modify the consistency of the formulation and add flavor.

The various ingredients were weighed and combined. The zinc citrate solution was added to these dry ingredients. Distilled water, Polysorbate-20, and Polysorbate-80 were then added to this mixture. The solution was heated until liquid, approximately 80° C. The solution was added to containers at 80° C. and allowed to cool to room temperature.

When essential oil is added to the formulation, the oil is added to the ingredient mixture and stirred at approximately 80° C. until the liquid has a uniform consistency. This solution is added to containers at 80° C. and allowed to cool to room temperature.

### Example 2

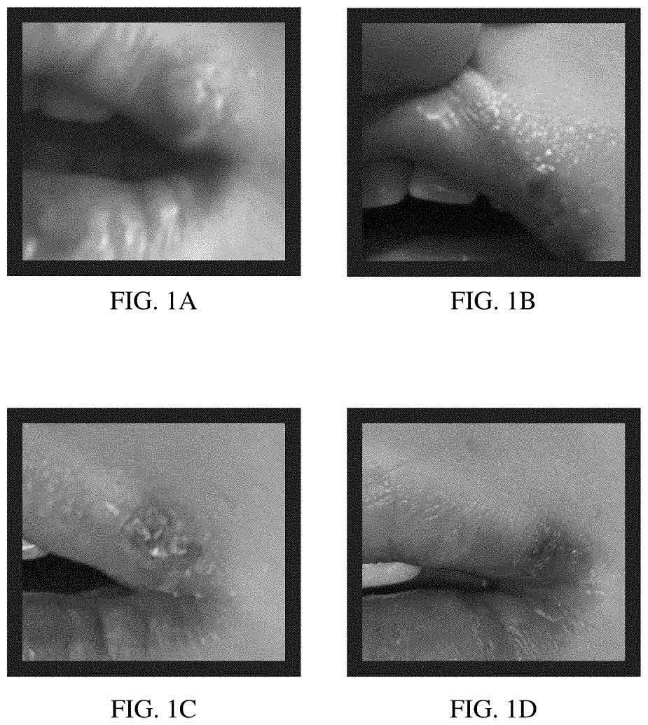

FIG. 1A shows an outbreak of blister-like lesions and sores on the mouth of a patient caused by Herpes Simplex Virus 1. The cream formulation from Table 4 was applied twice per day to the wound in an amount sufficient to cover the infected area and surrounding uninfected skin. Two hours after application (FIG. 1B), blistering was markedly reduced indicating a reduction in viral replication and reduced viral shedding. Some redness associated with immune response is still present. Symptoms of infection were absent after 14 hours (FIG. 1C). Scabs had formed over the area that had been previously infected, indicating that the lesions are healing, and redness associated with immune response is limited to areas immediately adjacent to the scabs. After 37 hours (FIG. 1D), scabs had fully formed and redness associated with immune response is very limited indicating that the infection is eliminated and the lesions are healing.

### Example 3

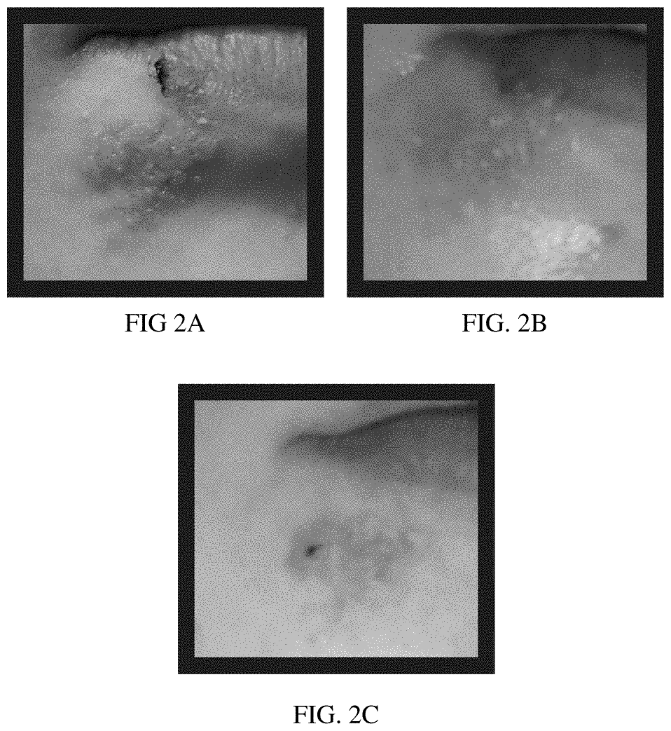

FIG. 2A shows an outbreak of blister-like lesions and sores on the mouth of a patient caused by Herpes Simplex Virus 1. The cream formulation from Table 4 was applied twice per day to the wound in an amount sufficient to cover the infected area and surrounding uninfected skin. Four hours after application (FIG. 2B), blistering was significantly reduced and some redness associated with immune response was present. Symptoms of infection were absent after 24 hours (FIG. 2C). Scabs had formed over the area that had been previously infected, indicating that the blisters are healing and viral shedding has stopped, while redness associated with immune response is limited to areas immediately adjacent to the scabs.

### Example 4

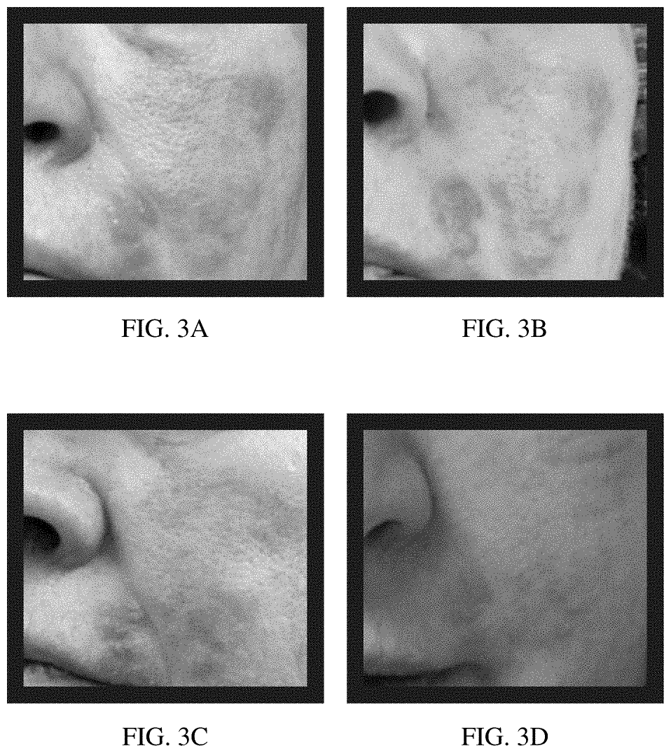

FIG. 3A shows an outbreak of zoster shingles on the skin of the patient's buccal region caused by Herpes Simplex Virus 2. The vesicular lesions formed small blisters filled with a serous exudate. The cream formulation from Table 4 was applied three times per day to the wound in an amount sufficient to cover the infected area and surrounding uninfected skin. Fifty-five hours after application (FIG. 3B), redness associated with the lesions had markedly decreased indicating a reduction in viral replication and reduced viral shedding. Seventy-two hours after application, (FIG. 3C), the cream formulation was applied twice daily to the wound. Blistering and pain associated with the infection was significantly reduced indicating a further reduction in viral replication and reduced viral shedding. Some redness associated with immune response is still present. Seven days after application (FIG. 3D), the cream formulation was applied once daily to the wound as needed for pain management. Redness and blistering associated with immune response is very limited indicating that the infection is eliminated and the lesions are healing.

1. A composition comprising a cannabinoid, a brassinosteroid, and a pharmaceutically acceptable excipient.

2. The composition of claim 1, wherein the concentration of cannabinoid is about 0.5 wt. % to about 50 wt. %, relative to the total amount of the composition.

3. The composition of claim 1, wherein the cannabinoid is cannabidiol, a cannabidiol analog, or combinations thereof.

4. The composition of claim 1, wherein the concentration of brassinosteroid is about 0.01 wt. % to about 5 wt. %, relative to the total amount of the composition.

5. The composition of claim 1, wherein the composition further comprises an anti-inflammatory compound, an antibiotic, a plant extract, a secondary antiviral agent, a mineral, a mineral salt, vitamins, amino acids, peptides, cyanobacteria, green algae, or combinations thereof.

6. The composition of claim 5, wherein the concentration of the amino acids, peptides, proteins, or combinations thereof is about 0.01 wt. % to about 5 wt. %, relative to the total amount of the composition.

7. The composition of claim 1, wherein the composition is a cream, lotion, salve, liniment, ointment, gel, paste, tonic, tincture, unguent, soap, shampoo, topical, oral pill, tablet, capsule, lip balm, or combinations thereof.

8. A method for treating viral infection comprising administering a therapeutically effective amount of a composition containing a cannabinoid, a brassinosteroid, and a pharmaceutically acceptable excipient to a patient in need of treatment.

9. The method of claim 9, wherein the viral infection is HSV-, HSV-2, or combinations thereof.

10. The method of claim 9, wherein administering comprises topically applying the composition to an affected area on the skin.

11. The method of claim 9, wherein administering comprises orally ingesting the composition.

12. The method of claim 9, wherein the concentration of cannabinoid is about 0.5 wt. % to about 50 wt. %, relative to the total amount of the composition.

13. The method of claim 9, wherein the cannabinoid is cannabidiol, a cannabidiol analog, or combinations thereof.

14. The method of claim 9, wherein the concentration of brassinosteroid is about 0.01 wt. % to about 5 wt. %, relative to the total amount of the composition.

15. The method of claim 9, wherein the composition further comprises an anti-inflammatory compound, an antibiotic, a plant extract, a secondary antiviral agent, a mineral, a mineral salt, vitamins, amino acids, peptides, cyanobacteria, green algae, or combinations thereof.

16. The method of claim 9, wherein the concentration of the amino acids, peptides, proteins, or combinations thereof is about 0.01 wt. % to about 5 wt. %, relative to the total amount of the composition.

17. The method of claim 9, wherein the composition is a cream, lotion, salve, liniment, ointment, gel, paste, tonic, tincture, unguent, soap, shampoo, topical, oral pill, tablet, capsule, lip balm, or combinations thereof.
